(19) United States
(12) Patent Application Publication
(10) Pub. No.: US 2019/0374323 A1
(43) Pub. Date: Dec. 12, 2019

(54) APPARATUS FOR IMPROVED EAR CANAL AIRFLOW IN DOGS

(71) Applicant: Tamara Dawn McKay, Cloverdale, CA (US)

(72) Inventor: Tamara Dawn McKay, Cloverdale, CA (US)

(21) Appl. No.: 16/004,821

(22) Filed: Jun. 11, 2018

(51) Int. Cl. A61D 99/00; A61F 11/00; A01K 27/00; A01K 13/00; A01K 25/00

(52) U.S. Cl. CPC A61D 99/00; A61F 11/00; A01K 25/00; A01K 13/006; A01K 27/001

(57) ABSTRACT

An apparatus for improved ear canal airflow in dogs is provided. The apparatus resembling a bonnet and includes a noseband, elastic fitting, collar portion, and a pair of ear pockets. The bonnet configured to fit over the head of a dog. The pair of ear pockets is configured to hold the dog ears, while fasteners are used to fold and secure the dog ears such that increased airflow enters the ear canals of the dog. The improved apparatus is used to treat and prevent ear infections in dogs.

### CROSS-REFERENCE TO RELATED APPLICATIONS

N/A

### BACKGROUND OF THE INVENTION

### 1. Field of the Invention

The present invention generally relates to animal devices, but more particularly to an apparatus for improved ear canal airflow in dogs.

### 2. Description of Related Art

It is well known that ear infections in dogs are a very common occurrence, and particularly painful for the dog. Common signs include scratching around the ear, and shaking the head. In most instances, ear infections occur when bacteria or yeast overgrows in the ear canal, usually from moisture entering the ear canal, or caused from long, floppy ears that lie flat against a dog's head covering the ear canal. This restricts air circulation creating a hypoxic environment, which is optimal for yeast and bacteria overgrowth, and, prohibits healing of an infected ear canal. Consequently, there is a need for an apparatus to treat and prevent ear infections in dogs, especially those with long, floppy ears.

### BRIEF SUMMARY OF THE INVENTION

In one embodiment of the present invention an apparatus for improved ear canal airflow in dogs is provided, comprising a bonnet shaped material having a front portion, a top portion, a left side portion, a right side portion, a back portion, and a bottom portion; an elastic fitting defining a first opening, wherein the elastic fitting is positioned at the front portion, extending from the top portion to the bottom portion; a collar portion defining a second opening, wherein the collar portion is positioned at the back portion, extending from the top portion to the bottom portion, the second and first openings configured to accept a muzzle, a head, and a neck area of a dog; a pair of ear pockets including a left ear pocket and a right ear pocket, each ear pocket having an upper portion and a lower portion, the upper portion on each ear pocket having an ear slit configured to accept an ear of the dog into each ear pocket; and, a mesh portion positioned on the lower portion of each ear pocket, the mesh portion sized to allow airflow but prevent unwanted objects from passing through.

In one embodiment, the top portion includes a first fastening member, the first fastening member orientated on the right side of the bonnet shaped material. In one embodiment, the upper portion of the pair of ear pockets includes a second and a third fastening member on the right ear pocket and the left ear pocket respectively. In one embodiment, the lower portion of the left ear pocket includes a fourth fastening member. In another embodiment, the third fastening member is configured to fasten to the first fastening member, such that a left dog ear is folded over the head of the dog, exposing a left ear canal allowing airflow to enter the left ear canal. In yet another embodiment, the second fastening member is configured to fasten to the fourth fastening member after the third fastening member is fastened to the first fastening member, such that a right dog ear is folded over the head of the dog, exposing a right ear canal allowing airflow to enter the right ear canal.

In one embodiment, the collar portion includes a plurality of collar loops configured to accept an adjustable collar. In another embodiment, a noseband configured to secure the apparatus to the muzzle is provided, wherein the noseband is portioned at a stop of the dog. In one embodiment, the noseband includes padding for pressure reduction. In one embodiment, each of the left and right side of the bonnet shaped material includes a reinforced slit configured to enable the noseband to pass through forming a throatlatch at a throat of the dog, the throatlatch adjustable via a securement clip, wherein the throatlatch secures the top portion of the bonnet shaped material from slipping while in use. In another embodiment, the throatlatch includes a tail portion having a clip. In yet another embodiment, the collar portion includes a plurality of collar loops configured to accept an adjustable collar, the adjustable collar including a ring configured to accept the clip of the throatlatch. In one embodiment, the bottom portion includes a sleeve configured to accept the tail portion. In one embodiment, the bonnet shaped material is constructed from a breathable, lightweight, and stretchable material.

### BRIEF DESCRIPTION OF THE SEVERAL VIEWS OF THE DRAWINGS

Other features and advantages of the present invention will become apparent when the following detailed description is read in conjunction with the accompanying drawings, in which:

FIG. 1 is a top view of apparatus for improved ear canal airflow in dogs according to an embodiment of the present invention;

FIG. 2 is a top view of apparatus for improved ear canal airflow in dogs with an ear pocket configured in an improved position according to an embodiment of the present invention;

FIG. 3 is a side view of the apparatus installed on a dog according to an embodiment of the present invention;

FIG. 4A is a side view of apparatus with the throatlatch secured to the collar ring according to an embodiment of the present invention; and,

FIG. 4B is a detail of FIG. 4A.

### DETAILED DESCRIPTION OF THE PREFERRED EMBODIMENT

The following description is provided to enable any person skilled in the art to make and use the invention and sets forth the best modes contemplated by the inventor of carrying out their invention. Various modifications, however, will remain readily apparent to those skilled in the art, since the general principles of the present invention have been defined herein to specifically provide an apparatus for improved ear canal airflow in dogs.

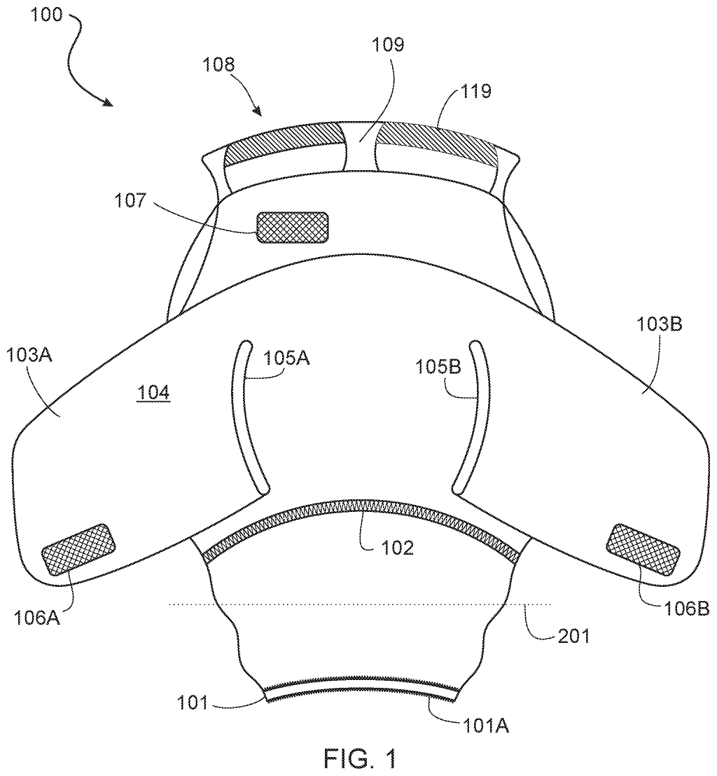

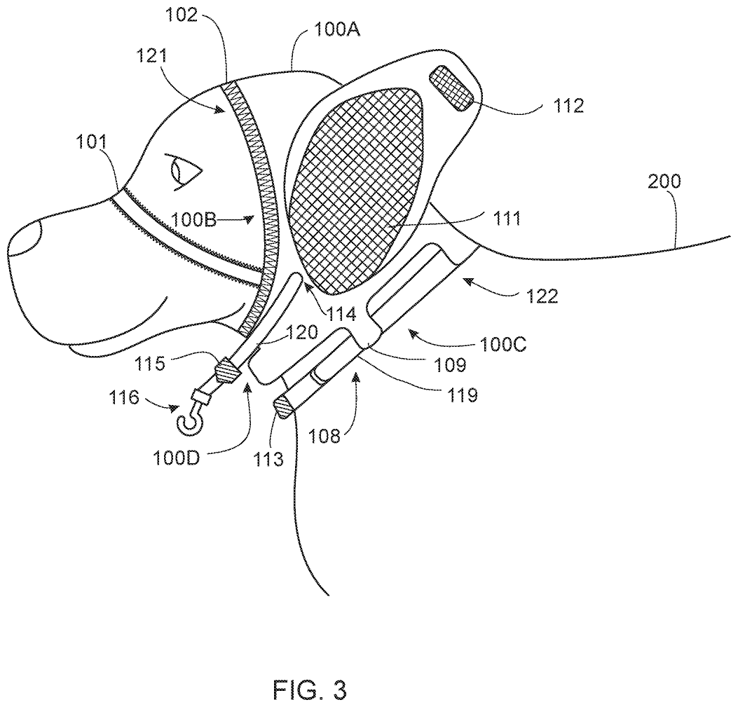

FIG. 1 is a top view of an apparatus 100 for improved ear canal airflow in dogs according to an embodiment of the present invention. Referring now to FIG. 1, the apparatus comprises a noseband 101, an elastic fitting 102, a pair of ear pockets 103A and 103B, and a collar portion 108. The noseband is configured to secure the apparatus to a muzzle of dog. In one embodiment, the noseband is configured to be positioned at just below the eyes at the stop, i.e. the degree of angle change between the skull and the nasal bone. In one embodiment, the noseband is constructed from and/or includes a padding 101A, such as a fleece material or similar for pressure reduction on the dog's muzzle. In one embodiment, the elastic fitting defines front portion of the apparatus, wherein the collar portion defines back portion of the apparatus, such that the material of the apparatus extends from the elastic fitting to the collar portion. The noseband extends independently from the material of the apparatus, such that a brow portion 201 of the dog is free from material, i.e. the dog's eyes are free from material coverage and obstructions (best seen in FIG. 3).

In one embodiment, the pair of ear pockets is configured to accept a pair of dog ears via a pair of ear slits 105A and 105B. The pair of ear pockets having an upper portion 104 including a pair of fasteners 106A and 106B symmetrically positioned in each back corner each respective ear pockets. In one embodiment, a single fastener 107 is positioned on a right, back portion of the apparatus in between the pair of ear pockets and the collar portion. In one embodiment, the collar portion includes collar loops 109, wherein an adjustable collar 119 is configured to thread the collar loops.

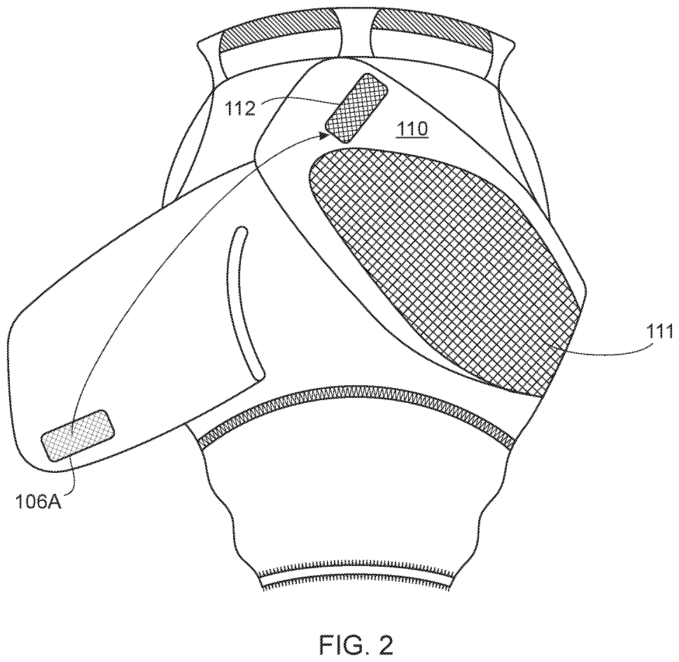

Now referring to FIG. 2, the left ear pocket of the pair of ear pockets is configured to be folded over the dog's head such that fastener 106B attaches to fastener 107, lifting the ear pocket exposing the lower portion 110 of the ear pocket, which exposes the ear canal. This is a particular advantage of the present invention, as the exposed ear canal allows for airflow to enter the ear canal drying out the ear, reducing moisture, and inhibiting the grow of yeast and bacteria, the primary causes of ear infections. In one embodiment, the lower portion of the ear pocket includes a mesh portion 111 configured to allow airflow but prevent insects, foxtails, and debris from entering the ear canal. Similarly, the right ear pocket of the pair of ear pockets is configured to folded over the dog's head via fastener 106A attaching to fastener 112 positioned on the lower portion of the left ear pocket. This configuration ensures the dog ears will not fold over into the dog's eyes. Although not illustrated, the lower portion of the right ear pocket includes a mesh portion identical to the mesh portion previously described. The fasteners may be any fasteners known in the art. In one embodiment, the fasteners are hook and loop fasteners, such as Velcro©.

Referring now to FIG. 3, the side view of the apparatus is illustrated, clearly showing its position on dog 200 when in use. The apparatus resembles a bonnet designed to slip over the dogs head, and fastens around the muzzle via noseband 101 as previously described. In one embodiment, a reinforced slit 114 is positioned on the side of apparatus, enabling the noseband to slip through the apparatus (represented as securement portion 120) from the inside surface (material surface not visible) to the outside surface (visible material surface). It is a particular advantage of the present invention that the noseband is adjustable and free to slide through the reinforced slit allowing for the noseband to have an optimal fit on varying muzzle circumferences and dog breeds. Although not explicitly illustrated in FIG. 3, the other side is essentially a mirror image, such that a similar securement portion 120 is configured to slide through a reinforced slit on the right side of the apparatus, wherein the two portions meet and join at securement clip 115, forming a throatlatch. In one embodiment, securement clip 115 enables a user to adjust the position of the securement clip along the throatlatch such that it may be relatively snug under the dog's throat/chin area. This secures the top portion 100A of the apparatus from slipping up, while also keeping the noseband secure, yet not pulled back to interfere with the movement of the mouth.

Still referring to FIG. 3, elastic fitting 102 defines a first opening 121, wherein the elastic fitting is positioned at a front portion 100B, extending from top portion 100A to a bottom portion 100D. The collar portion 108 defines a second opening 122, wherein the collar portion is positioned at a back portion 100C, extending from the top portion to the bottom portion. The first and second openings configured to accept the muzzle, head, and a neck area of a dog.

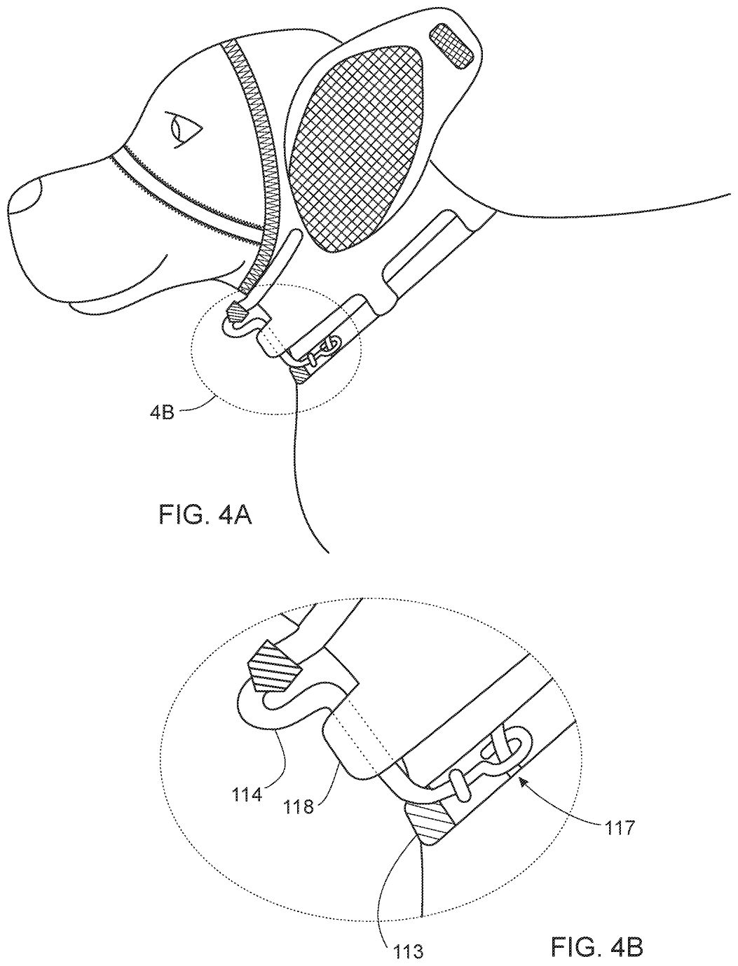

Referring now to FIG. 4, a tail portion 114 is configured to enter through a sleeve 118 positioned on the bottom portion of the apparatus between the elastic fitting and the collar portion. Next, clip 116 may be fastened to a collar ring 117 to prevent the apparatus from slipping. Specifically, the clip attaches to the collar ring to prevent the top portion of the material from slipping while in use.

In one embodiment, an existing (previously owned) adjustable collar may be used, wherein the exiting adjustable collar is threaded through collar loops positioned on the lower, back portion of the apparatus. In an alternative embodiment, the adjustable collar can be provided with the apparatus. In both instances, a collar adjustment means 113 is required, enabling the circumference of the adjustable collar to be adjusted such that the collar may fit a variety of neck sizes.

In a preferred embodiment, the apparatus is provided in a few specific sizes, such as small, medium, and large. In each specific size, the adjustability of the design as previously described will accommodate most dog breads, including varies head sizes and shapes. The constructed material of the apparatus may be any known material in the art, which is breathable, lightweight, and stretchable, including but not limited to cotton, nylon, polyester, polypropylene, spandex, cellulosic fiber, bamboo, or a combination thereof.

Although the invention has been described in considerable detail in language specific to structural features and or method acts, it is to be understood that the invention defined in the appended claims is not necessarily limited to the specific features or acts described. Rather, the specific features and acts are disclosed as exemplary preferred forms of implementing the claimed invention. Stated otherwise, it is to be understood that the phraseology and terminology employed herein, as well as the abstract, are for the purpose of description and should not be regarded as limiting. Therefore, while exemplary illustrative embodiments of the invention have been described, numerous variations and alternative embodiments will occur to those skilled in the art. Such variations and alternate embodiments are contemplated, and can be made without departing from the spirit and scope of the invention.

It should further be noted that throughout the entire disclosure, the labels such as left, right, front, back, top, bottom, forward, reverse, clockwise, counter clockwise, up, down, or other similar terms such as upper, lower, aft, fore, vertical, horizontal, oblique, proximal, distal, parallel, perpendicular, transverse, longitudinal, etc. have been used for convenience purposes only and are not intended to imply any particular fixed direction or orientation. Instead, they are used to reflect relative locations and/or directions/orientations between various portions of an object.

In addition, reference to “first,” “second,” “third,” and etc. members throughout the disclosure (and in particular, claims) are not used to show a serial or numerical limitation but instead are used to distinguish or identify the various members of the group.

What is claimed is:

1. An apparatus for improved ear canal airflow in dogs, comprising:
a bonnet shaped material having a front portion, a top portion, a left side portion, a right side portion, a back portion, and a bottom portion;
an elastic fitting defining a first opening, wherein the elastic fitting is positioned at the front portion, extending from the top portion to the bottom portion;
a collar portion defining a second opening, wherein the collar portion is positioned at the back portion, extending from the top portion to the bottom portion, the second and first openings configured to accept a muzzle, a head, and a neck area of a dog;
a pair of ear pockets including a left ear pocket and a right ear pocket, each ear pocket having an upper portion and a lower portion, the upper portion on each ear pocket having an ear slit configured to accept an ear of the dog into each ear pocket; and,
a mesh portion positioned on the lower portion of each ear pocket, the mesh portion sized to allow airflow but prevent unwanted objects from passing through.

2. The apparatus for improved ear canal airflow in dogs of claim 1, wherein the top portion includes a first fastening member, the first fastening member orientated towards the right side of the bonnet shaped material.

3. The apparatus for improved ear canal airflow in dogs of claim 2, wherein the upper portion of the pair of ear pockets includes a second and a third fastening member on the right ear pocket and the left ear pocket respectively.

4. The apparatus for improved ear canal airflow in dogs of claim 3, wherein the lower portion of the left ear pocket includes a fourth fastening member.

5. The apparatus for improved ear canal airflow in dogs of claim 4, wherein the third fastening member is configured to fasten to the first fastening member, such that a left dog ear is folded over the head of the dog, exposing a left ear canal allowing airflow to enter the left ear canal.

6. The apparatus for improved ear canal airflow in dogs of claim 5, wherein the second fastening member is configured to fasten to the fourth fastening member after the third fastening member is fastened to the first fastening member, such that a right dog ear is folded over the head of the dog, exposing a right ear canal allowing airflow to enter the right ear canal.

7. The apparatus for improved ear canal airflow in dogs of claim 1, wherein the collar portion includes a plurality of collar loops configured to accept an adjustable collar.

8. The apparatus for improved ear canal airflow in dogs of claim 1, further comprising a noseband configured to secure the apparatus to the muzzle, wherein the noseband is portioned at a stop of the dog.

9. The apparatus for improved ear canal airflow in dogs of claim 8, wherein the noseband includes padding for pressure reduction.

10. The apparatus for improved ear canal airflow in dogs of claim 8, wherein each of the left and right side of the bonnet shaped material includes a reinforced slit configured to enable the noseband to pass through forming a throatlatch at a throat of the dog, the throatlatch adjustable via a securement clip, wherein the throatlatch secures the top portion of the bonnet shaped material from slipping while in use.

11. The apparatus for improved ear canal airflow in dogs of claim 10, wherein the throatlatch includes a tail portion having a clip.

12. The apparatus for improved ear canal airflow in dogs of claim 11, wherein the collar portion includes a plurality of collar loops configured to accept an adjustable collar, the adjustable collar including a ring configured to accept the clip of the throatlatch.

13. The apparatus for improved ear canal airflow in dogs of claim 12, wherein the bottom portion includes a sleeve configured to accept the tail portion.

14. The apparatus for improved ear canal airflow in dogs of claim 1, wherein the bonnet shaped material is constructed from a breathable, lightweight, and stretchable material.
